(19) United States
(12) Patent Application Publication
(10) Pub. No.: US 2006/0211703 A1
(43) Pub. Date: Sep. 21, 2006

(54) Substituted alkyl-pyridazinones for the treatment of memory and learning malfunctions

(76) Inventors: Gyorgy Levay, Budakeszi (HU); Istvan Gacsalyi, Budapest (HU); Bernadett Marko, Budapest (HU); Eva Schmidt, Budapest (HU); Andras Egyed, Budapest (HU); Hajnalka Kompagne, Budapest (HU); Csilla Leveleki, Budapest (HU); Aniko Miklosne Koivacs, Budapest (HU); Gabor Szenasi, Budapest (HU); Janos Wellmann, Budapest (HU); Laszlo Gabor Harsing, Budapest (HU); Jozsef Barkoczy, Budapest (JP); Gyula Simig, Budapest (HU); Peter Kotay Nagy, Vac (HU)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/535,039

(22) PCT Filed: Nov. 13, 2003
(86) PCT No.: PCT/HU03/00096

§ 371 Date: May 3, 2006

(30) Foreign Application Priority Data
Nov. 13, 2003 (HU) P0203929

(51) Int. Cl. A61K 31/501

(52) U.S. Cl. 514/252.01; 514/252.02

(57) ABSTRACT

The compounds of the general Formula I, wherein R¹ stands for hydrogen or lower alkyl; one of symbols X and Y stands for hydrogen or halogen and the other represents a group of the general Formula II, R² is hydrogen or lower alkyl; n is 1, 2 or 3; R³ is hydrogen, lower alkyl or aryl-lower alkyl; Z is -0-; or R³ and Z together with the intermediate atoms form piperazino ring; Q and W independently from each other stands for —CH═ or —N═; and R⁴, R⁵ and R⁶ can be the same or different and stand for hydrogen, halogen, trifluoromethyl or lower alkoxy; or R⁴ and R⁵ together form an ethylenedioxy group) and pharmaceutically acceptable salts thereof can be used for the treatment or prophylaxis of malfunctions of memory and/or cognitive decline or prevention of decline of learning abilities.

### FIELD OF THE INVENTION

The present invention relates to the use of substituted alkyl-pyridazinone derivatives for the treatment of malfunctions of memory and/or cognitive decline or prevention of decline of learning abilities.

The present invention also relates to preparation of pharmaceutical composition for the treatment of the above-mentioned diseases, disorders and conditions.

### TECHNICAL BACKGROUND

The piperazinyl-alkyl-3(2H)-pyridazinone derivatives claimed in the patent application No EP 372 305 possess antihypertensive effects and are applicable as treatment of cardiac insufficiency and peripheral circulation disturbances.

The alkyl-pyridazinone derivatives claimed in the Hungarian Patent Application No 01/03912 have anxiolytic effects and are applicable as active anxiolytic ingredients.

It has been found that the alkyl-pyridazinone derivatives disclosed in the Hungarian Patent Application No 01/03912 are useful in further indications different from anxiety, cardiovascular and heart diseases.

The literature discusses two basic type of memory. In case of the so-called short-term memory, which is, one of the two types, the information learned is saved from minutes to hours. In case of the other type, referred to as long-term memory, the engram can be saved from hours to years [Baddley and Warrington J. Verb, Learn. Verb Behav. 9, 176-179 (1970); Wright et al. Science 229, 287-289 (1985)].

The process of transferring the information from short-term memory to long-term memory is referred as memory consolidation.

The process of manifestation or retrieval of the fixed information from the short or long-term memory is referred as recall.

The total amnesia is relatively rare, however we have to face more and more with the increasing prevalence of diseases accompanied by memory deficits. At present, 18 million people suffer from Alzheimer disease and if we consider only this disease, this number will doubled in the next 25 years [Fletcher, Mol. Med. Today, 3/10 p. 429-434 (1997)].

### DISCLOSURE OF THE INVENTION

The subject of the invention is to develop new pharmaceutical products for the effective use for the treatment of diseases or conditions accompanied with memory malfunctions.

The above subject is reached by means of the present invention in a surprising way.

The invention is based on the recognition that the compounds disclosed in Hungarian Patent Application No 01/03912 possess stimulating effects on cognitive processes (memory, thinking, attention, etc.).

The present invention is directed to the use of compounds of the general Formula 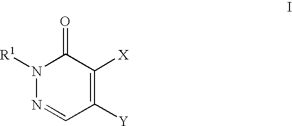
(wherein
R¹ stands for hydrogen or lower alkyl;
one of symbols X and Y stands for hydrogen or halogen and the other represents a group of the general Formula 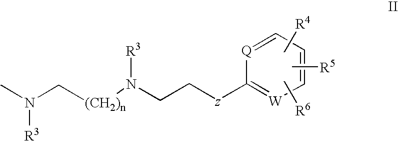
R² is hydrogen or lower allyl;
n is 1, 2 or 3;
R³ is hydrogen, lower allyl or aryl-lower alkyl;
Z is —O—; or
R³ and Z together with the intermediate atoms form a piperazino ling;
Q and W independently from each other stands for —CH═ or —N═; and
R⁴, R⁵ and R⁶ can be the same or different and stand for hydrogen, halogen, trifluoromethyl or lower alkoxy; or R⁴ and R⁵ together form an ethylenedioxy group)
   and salts thereof for the preparation of pharmaceutical compositions for the treatment or prophylaxis of malfunctions of memory and/or cognitive decline or prevention of decline of learning abilities.

According to a preferred embodiment of the present invention the compounds of the general Formula I and pharmaceutically acceptable salts thereof are used for the preparation of pharmaceutical compositions for the treatment or prophylaxis of Korsakoff syndrome, Alzheimer disease, Huntington syndrome or Parkinson disease and/or mental decline due to ageing processes or impairment of the cognitive functions due to exposure to toxical substances.

### DETAILED DESCRIPTION OF THE INVENTION

The definition of the terms used in the present patent specification is to be interpreted as follows:

The term “lower alkyl” stands for straight or branched chain alkyl group containing 1-6, preferably 1-4 carbon atoms (e.g. methyl, ethyl, n-propyl, isopropyl, n-butyl, secondary butyl, tertiary butyl etc.).

The term “halogen” encompasses fluorine, chlorine, bromine and iodine atom and stands preferably for chlorine or bromine, particularly for chlorine.

The term “lower alkoxy” stands for alkyl group defined as above attached through an oxygen atom (e.g. methoxy, ethoxy, n-propoxy etc.).

The term “aryl-lower alkyl” stands for lower alkyl groups defined as above substituted by an aryl group (e.g. phenyl, naphthyl etc.). The aryl-lower alkyl group can be e.g. benzyl, β-phenyl-ethyl or β,β-diphenyl-ethyl etc.).

The term “pharmaceutically acceptable acid addition salts” relates to salts formed with inorganic or organic acids which are suitable for medical use. For salt formation e.g. hydrochloride, hydrogen bromide, sulfuric acid, phosphoric acids formic acid, acetic acid, maleic acid, fumaric acid, lactic acid, tartaric acid, succinic acid, citric acid, methanesulfonic acid, benzenesulfonic acid, p-toluenesulfonic acid etc. can be used.

As already mentioned above, the compounds of the general Formula I exhibit anxiolytic effect without exerting any sedative side effect to a substantial amount. The above recognition is surprising and could not be foreseen because from the anxiolytic effect one cannot draw conclusion to a favourable effect exerted on the cognitive functions; these are disease categories being completely different from the pharmaceutical point of view. Moreover, anxiolytics are known to have a memory destroying effect as an undesirable side effect. On the other hand, we have found in a surprising way that the compounds of the general Formula I do no exhibit only anxiolytic activity but additionally increase the learning procedure and the memory as well.

According to a preferred embodiment of the present invention as active ingredient compounds of the general Formula I and pharmaceutically acceptable salts thereof are used in which
R¹ is hydrogen, methyl, ethyl or tertiary butyl;
one of symbols X and Y is hydrogen or chlorine and the other represents a group of the general Formula II;
R² is hydrogen or methyl;
n is 1 or 2;
R³ is hydrogen, methyl or benzyl;
Z is —O—; or
R³ and Z together with the intermediate atoms form a piperazino ring;
R⁴, R⁵ and R⁶ can be the same or different and stand for hydrogen or halogen; or R⁴ and R⁵ together form an ethylenedioxy group); and
Q and W stand for —CH═.

According to a particularly preferred embodiment of the present invention as active ingredient one of the following compounds of the general Formula I of a pharmaceutically acceptable acid addition salt thereof is used:
4-(3-((2-(2,3-dihydro-benzo[1,4]dioxine-5-yloxy)-ethyl)-methyl-amino)-propyl-amino)-5-chloro-2H-pyridazine-3-one;
4-(3-{[2-(2,3-dihydro-benzo[1,4]dioxine-5-yloxy)-ethyl]-propyl-amino}-propyl-amino)-5-chloro-2H-pyridazine-3-one;
4-(3-(benzyl-(2-(2,3-dihydro-benzo[1,4]dioxine-5-yloxy)-ethyl)-amino)-propyl-amino)-5-chloro-2H-pyridazine-3-one;
4-(4-(4-(2,3-dihydro-benzo[1,4]dioxine-5-yl)-piperazine-1-yl)-buthylamino)-5-chloro-2H-pyridazine-3-one;
5-(2-(4-(2,3-dihydro-benzo[1,4]dioxine-5-yl)-piperazine-1-yl)-ethylamino)-4-chloro-2H-pyridazine-3-one;
4-chloro-5-(2-(4-(2,3-dihydro-1,4-benzodioxine-5-yl)-piperazine-1-yl)-ethylamino)-2-methyl-2H-pyridazine-3-one;
4-chloro-5-((2-(4-(2,3-dihydro-benzo[1,4]dioxine-5-yl)-piperazine-1-yl)-ethyl)-methyl-amino-2H-pyridazine-3-one;
2-tert.-buthyl-5-chloro-4-(2-(4-(2,3-dihydro-benzo[1,4]dioxine-5-yl)-piperazine-1-yl)-ethylamino)-2H-pyridazine-3-one;
4-(3-(2-(2,3-dihydro-benzo[1,4]dioxine-5-yloxy)-ethylamino)-propylamino)-2H-pyridazine-3-one;
5-{2-[4-(2,3-dihydro-1,4-benzodioxine-5-yl)-piperazine-1-yl]-ethylamino}-2H-pyridazine-3-one;
5-{2-[4-(7-chloro-2,3-dihydro-benzo[1,4]dioxine-5-yl)-piperazine-1-yl]-ethylamino}-2H-pyridazine-3-one;
5-{3-[4-(2,3-dihydro-1,4-benzodioxine-5-yl)-piperazine-1-yl]-propylamino}-2H-pyridazine-3-one;
5-(2-(2-(2,3-dihydro-benzo[1,4]dioxine-5-yloxy)-ethylamino)-ethylamino)-2H-pyridazine-3-one;
5-{2-[4-(2,3-dihydro-1,4-benzodioxine-5-yl)-piperazine-1-yl]-ethylamino}-2-methyl-2H-pyridazine-3-one;
5-({2-[4-(2,3-dihydro-benzo[1,4]dioxine-5-yl)-piperazine-1-yl]-ethyl})-methyl-amino)-2H-pyridazine-3-one and monohydrate thereof;
5-(2-(4-(2,3-dihydro-benzo[1,4]dioxine-5-yl)piperazine-1-yl)-ethyl-methylamino)-2-methyl-2H-pyridazine-3-one;
5-({2-[4-(2,3-dihydro-benzo[1,4]dioxine-5-yl)-piperazine-1-yl]-ethyl}-methyl-amino)-4-chloro-2-methyl-2H-pyridazine-3-one;
5-(2-{benzyl-[2-(2,3-dihydro-benzo[1,4]dioxine-5-yloxy)-ethyl]-amino}-ethylamino)-4-chloro-2-methyl-2H-pyridazine-3-one;
5-{2-[2-(2,3-dihydro-benzo[1,4]dioxine-5-yloxy)-ethylamino]-ethyl-amino}-2-methyl-2H-pyridazine-3-one;
5-{2-[4-(methoxy-trifluoromethyl-phenyl)-piperazine-1-yl]-ethylamino}-2H-pyridazine-3-one;
5-(2-[4-(2-fluoro-phenyl)-piperazine-1-yl]-ethylamino}-2H-pyridazine-3-one;
5-(2-[4-phenyl-piperazine-1-yl]-ethylamino}-2H-pyridazine-3-one;
5-[2-(4-pyridine-2-yl-piperazine-1-yl)-ethylamino]-2H-pyridazine-3-one;
5-[2-(4-pyrimidine-2-yl-piperazine-1-yl)-ethylamino]-2H-pyridazine-3-one;
5-{2-[4-(3-chloro-phenyl)-piperazine-yl]-ethylamino}-2H-pyridazine-3-one; and
5-{2-[4-(4-fluor-phenyl)-piperazine-1-yl]-ethylamino}-2H-pyridazine-3-one.

The preparation of the compounds of the general Formula I is disclosed in Hungarian patent application 01/03912.

Thus the compounds of the general Formula I can be prepared e.g. by
a) for the preparation of compounds of the general Formula I wherein X is hydrogen or halogen and Y stands for a group of the general Formula II, reacting a compound of the general Formula 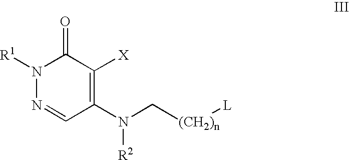
with a compound of the general Formula 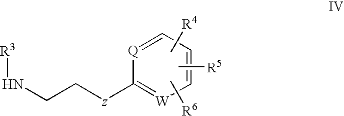
or
b) for the preparation of compounds of the general Formula I wherein X stands for a group of the general Formula II and Y stands for hydrogen or halogen, reacting a compound of the general Formula 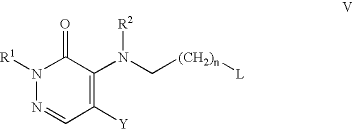
with a compound of the general Formula IV; or
c) for the preparation of compounds of the general Formula I wherein X stands for hydrogen or halogen and Y stands for a group of the general Formula II, reacting a compound of the general Formula 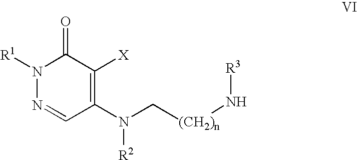
with a compound of the general Formula 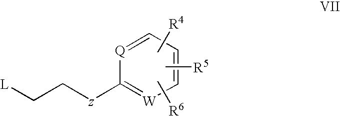
or
d) for the preparation of compounds of the general Formula I wherein X stands for a group of the general Formula II and Y stands for hydrogen or halogen, reacting a compound of the general Formula 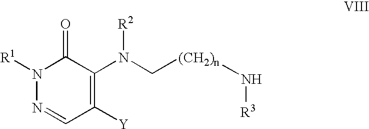
with a compound of the general Formula VII; or
e) for the preparation of compounds of the general Formula I wherein one of symbols X and Y stands for hydrogen or halogen and the other represents a group of the general Formula II, reacting a dihalogeno compound of the general Formula 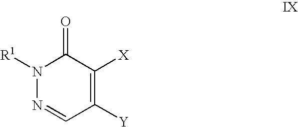
(wherein X and Y stands for halogen) with a compound of the general Formula 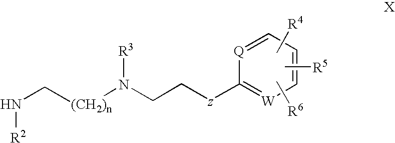
   and if desired converting the compound of the general Formula I thus obtained in which one of symbols X and Y stands for halogen and the other represents a group of the general Formula II by catalytic dehalogenation into the corresponding compound of the general Formula I in which either X stands for hydrogen and Y represents a group of the general Formula II or X stands for a group of the general Formula II and Y represents hydrogen;
   and if desired converting a compound of the general Formula I into a pharmaceutically acceptable acid addition salt thereof.

The above processes a), b), c), d) and e) can be carried out by methods analogous to those disclosed in prior art, see e.g. March, J.: Advanced Organic Chemistry, Reactions, mechanism and structure, 4^{th} Edition, John Wiley & Sons, New York, 1992.

According to process e) mostly a mixture of compounds of the general Formula I is formed. Thus depending on the starting materials used, a mixture of two compounds of the general Formula I is formed, in which X stands for a group of the general Formula II and Y represents halogen, and X stands for halogen and Y represents a group of the general Formula II, respectively. The mixture thus obtained can be separated into the components by known methods of preparative organic chemistry, e.g. fractioned crystallization.

In case of subjecting a compound of the general Formula I, wherein X or Y stands for halogen, preferably chlorine, to catalytic hydrogenation, dehalogenation takes place and the corresponding compound of the general Formula I is formed in which X or Y stand for hydrogen.

Catalytic hydrogenation can be carried out by methods known from prior art, e.g. March, J.: Advanced Organic Chemistry, Reactions, mechanism and structure, 4^{th} Edition, John Wiley & Sons, New York, 1992. As hydrogen source e.g. hydrogen gas, hydrazine, hydrazine hydrate, formic acid, trialkyl ammonium formate or alkali formate can be used. The catalyst may be preferably palladium, platinum oxide or Raney-nickel.

The reaction can be carried out in the presence or absence of an acid binding agent. For this purpose an inorganic base (e.g. sodium hydroxide) or an organic base (e.g. hydrazine, triethyl amine, diisopropyl-ethyl-amine etc.) can be used. The reaction can be performed in an inert protic or aprotic solvent or a mixture thereof. As protic solvent e.g. an alkanol, water or a mixture thereof can be used, while as aprotic solvent preferably dioxane or dichloro methane can be applied. The reaction temperature is generally between 0-150° C., preferably 20-100° C.

The compound of the general Formula I can be converted into the acid addition salt and the base of the Formula I can be set free from an acid addition salt in a manner known per se.

The alkylamino-pyridazinone derivatives of the general Formula III and V can be prepared as described in PCT/HU98/00054.

The amines of the general Formula IV used as starting material are partly known compounds. The new compounds of the general Formula IV can be prepared in an analogous manner [Pollard et al, J. Am. Chem. Soc., 56, 2199 (1934)].

The aminoalkylamino-pyridazinone derivatives of the general Formulae VI and VIII are also partly known from prior art. The new compounds can be prepared by an analogous method described in prior art [Haerer et al, Arzneim. Forsch., 39(6), 714-716 (1989)].

The starting materials of the general Formula VII are also partly known. The new compounds can be prepared by methods known per se [Augstein, J. et al, J. Med. Chem., 8, 356-367 (1965)].

The dihalogeno-pyridazinone derivatives of the general Formula IX are partly known. The new compounds can be prepared by known methods [Homer et al, J. Chem. Soc., 1948, 2194].

The compounds of the general Formula X can be prepared from the compounds of the general Formula IV by methods known per se [Shigenaga, S. et al, Arch. Pharm., 329(1), 3-10 (1996); Janssens, F. et al, J. Med. Chem., 28 (12), 1934-1943 (1985); He Xiao Shu et al, Bioorg. Med. Chem. Lett., 7(18), 2399-2402 (1997)].

According to a further aspect of the present invention there is provided a process for the preparation of pharmaceutical compositions containing as active ingredient a compound of the general Formula I or a pharmaceutically acceptable acid addition salt thereof which comprises admixing the active ingredient prepared by known method with conventional pharmaceutical carriers and/or excipients and finishing the mixture in pharmaceutical compositions suitable for the treatment or prophylaxis of malfunctions of memory and/or cognitive decline or prevention of decline of learning abilities.

According to a preferred embodiment of the present invention pharmaceutical compositions are prepared suitable for the treatment or prophylaxis of Korsakoff syndrome, Alzheimer disease, Huntington disease or Parkinson disease and/or mental decline due to ageing processes or impairment of the cognitive functions due to exposure to toxical substances.

According to a favourable aspect of the present invention there are provided pharmaceutical compositions for the treatment or prophylaxis of malfunctions of memory and/or cognitive decline or prevention of decline of learning abilities comprising as active ingredient a compound of the general Formula I or a pharmaceutically acceptable acid addition salt thereof in admixture with suitable inert, solid or liquid pharmaceutical carriers and/or auxiliary agents.

According to a preferred embodiment of the present invention pharmaceutical compositions are prepared suitable for the treatment or prophylaxis of Korsakoff disease, Alzheimer disease, Huntington syndrome or Parkinson disease and/or mental decline due to ageing processes or impairment of the cognitive functions due to exposure to toxical substances.

The pharmaceutical compositions according to the present invention contain generally 0.1-95% by weight, preferably 1-50% by weight, particularly preferably 5-30% by weight of the compound of the general Formula I or a pharmaceutically acceptable acid addition salt thereof.

The pharmaceutical composition can be administered orally, parenterally, rectally or transdermally or can be used locally. The pharmaceutical compositions can be solid or liquid.

The oral solid pharmaceutical compositions may be powders, capsules, tablets, film-coated tablets, microcapsules etc. and can contain as carrier e.g. binders (such as gelatine, sorbitol, polyvinyl pyrrolidone etc.), fillers (e.g. lactose, glucose, starch, calcium phosphate etc.), tabletting auxiliary agents (e.g. magnesium stearate, talc, polyethylene glycol, silicium dioxide etc.), wetting agents (e.g. sodium lauryl sulfate) etc.

The oral liquid pharmaceutical compositions may be in the form of solutions, suspensions and emulsions and can contain as carrier e.g. suspending agents (e.g. gelatine, carboxymethyl cellulose etc.), emulsifiers (e.g. sorbitan monooleate etc.), solvents (e.g. water, oil, glycerine, propylene glycol, ethanol), stabilizers (e.g. p-hydroxy-benzene-methyl or propyl ester) etc.

The parenterally administrable pharmaceutical compositions are generally sterile solutions of the active ingredient.

The above dosage forms are mentioned only in an exemplifying non-limiting character and are known per se [see e.g. the Manual Remington's Pharmaceutical Sciences, 18^{th} edition, Mack Publishing Co., Easton, USA (1990)].

The pharmaceutical compositions of the present invention can be prepared by known methods of pharmaceutical industry. Thus one may proceed by admixing the active ingredient with one or more carriers and finishing the mixture thus obtained in a form suitable for medical use in a manner known per se. The above methods are known from prior art, e.g. the above manual Remington's Pharmaceutical Sciences.

According to a further aspect of the present invention there is provided the use of a compound of the general Formula I or a pharmaceutically acceptable acid addition salt thereof for the treatment or prophylaxis of malfunctions of memory and/or cognitive decline or prevention of decline of learning abilities.

According to a preferable embodiment of the above aspects the compounds of the general Formula I and pharmaceutically acceptable acid addition salts thereof are used for the treatment or prophylaxis of Korsakoff syndrome, Alzheimer disease, Huntington syndrome or Parkinson disease and/or mental decline due to ageing processes or impairment of the cognitive functions due to exposure to toxical substances.

According to a further feature of the present invention there is provided a process for the treatment or prophylaxis of malfunctions of memory and/or cognitive decline or prevention of decline of learning abilities which comprises administering to the patient in need of such treatment a pharmaceutically efficient amount of a compound of the general Formula I or a pharmaceutically acceptable acid addition salt thereof.

According to a preferred embodiment of the above aspect there is provided a process for the treatment or prophylaxis of Korsakoff syndrome, Alzheimer disease, Huntington syndrome or Parkinson disease and/or mental decline due to ageing processes or impairment of the cognitive functions due to exposure to toxical substances which comprises administering to the patient in need of such treatment a pharmaceutically efficient amount of a compound of the general Formula I or a pharmaceutically acceptable acid addition salt thereof.

The compounds of the general Formula (I) as we have already mentioned, possess considerable anxiolytic property without sedative side effects in its anxiolytic dose range.

The recognition according to the present invention is unforeseen and non-obvious because the effect on the cognitive function is not a result of the anxiolytic effect. From therapeutical point of view, the anxiolytic effect and the effect on the cognitive function, are completely different disease categories. Moreover, anxiolytics, such as 1,4 benzodiazepines, are characterized by memory impairing effects as unwanted side effects. In contrast, we have surprisingly found that the compounds of general Formula (I) besides their anxiolytic efficacy improve either the learning processes or the memory.

The improving effects on the learning and memory processes of the compounds of general Formula (I) were verified by the following experiments:

Method

Male Wistar rats weighing 200-220 g were used. The animals were obtained from Charles River Co. They were kept in a room with normal 12-12 h light dark cycle (light on: 06:00) at relative humidity of 60±10%.

The experiment was performed in a five-channel “step through”-type passive avoidance learning apparatus. The equipment consisted of to adjacent Plexi-glass box of 20×20×16 cm. One of them was made of regular transparent Plexi-glass and the other one was made of black, non-transparent Plexi-glass. The boxes were connected with a 7.5×8 cm passageway, equipped with a computer-controlled guillotine-door. The passage of the rats through the door was detected by infrared photocells arranged in two parallel lines in the opening of the passageway. The door was automatically closed when the animals passed through. The dark compartment was equipped with stainless steel grid floor through which electric foot shocks could have been delivered to the animals. A 10 W light bulb was installed above the passage way in the light compartment.

The experiment was performed on two consecutive days, in two sessions, which were 24 h apart from each other.

On Day 1 (Acquisition) the animals acquired information about the situation (grid floor shock in the dark compartment), on Day 2 (Retention) they recalled the acquired information to avoid punishment (“if I go into the dark I will be punished, so I stay outside in the light”.

Day 1 (Acquisition)

The individually numbered animals were placed into the light compartment of the equipment. After 30 s the guillotine door was opened and the rats could have freely passed to the dark (considered as safe) compartment. Step through latency was automatically determined. (Step-through latency is the time period spanning from door opening to the time when the animal passed into the dark compartment.) The door was closed then, and the timer was automatically stopped. An electric foot shock of 1.2 mA lasting 2.5 s was applied to the animal trough the grid floor 3 s after the door has been closed, except for rats in the absolute control group (no shock+vehicle treated). Test animals were removed from the dark compartment immediately after foot shock has been delivered. The function of the absolute control group was to show that shocked animals will remember to the unpleasant foot shock as revealed by increased latency time when compared to absolute control. That is the essence of acquisition.

Day 2 (Retention)

After 24 h the animals were placed again in the light compartment of the test apparatus and step-through latency was measured as described at Acquisition day, except that no foot shock was applied to the animals in any group on the second day. A maximum of 180 s time interval was available for the rats to pass into the dark compartment. The animals were removed from the light compartment if they did not pass to the dark compartment within the 180 s test period.

Treatments

When effect on the acquisition was studied, the 5-[2-[4-(2,3-dihydro-benzo[1,4]dioxine-5-yl)-piperazine-1-yl]-ethylamine]-2H-pyridazine-3-one (further compound A) in a dose of 1 mg/kg ip. or vehicle (0.4% methylcellulose) was administered in a volume of 1 ml/kg at Day 1, 30 min before placing the animals into the apparatus.

When the effects on recall was studied (long term memory), the treatments in a dose of 1 mg/kg ip, in a volume of 1 ml/kg, were performed at Day 2, 30 minutes before placing the animals into the apparatus.

Statistical analysis was performed by multiple analysis of ANOVA, followed by post hoc Duncan-test for significant differences between groups.

Discussion

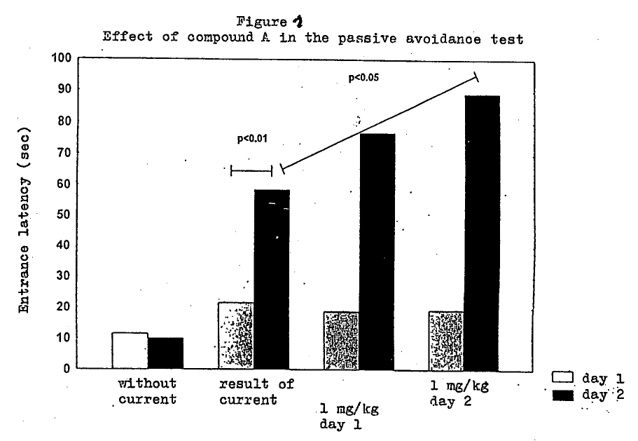

The experimenters surprisingly found that compound A significantly increased step-through latency into the dark compartment of the passive avoidance apparatus both after Day 1 and Day 2 administration of the compound (FIG. 1).

It is shown in FIG. 1 that in the absolute control group (no shock, untreated), the step-trough latency was approximately the same on both experimental days (meaning that there was nothing to recall and avoid on the second day for this treatment group).

In the shocked, vehicle treated control group the unavoidable foot shock resulted in significantly increased step-through latency in Day 2 when compared to absolute control. The experimental animals recalled the annoying experience (foot shock) in the dark, therefore, they pass into the dark compartment after significantly longer time (increased latency).

In the experimental groups, where the animals were treated with compound A (1 mg/kg ip.), this augmented latency has been further increased by both type of treatment (Day 1 or Day 2). This means that animals of these groups either learned faster (after treatment in Day 1) or they remembered better (after treatment in Day 2) to the electrical shock applied in the Day 1. The effect was statistically significant after the Day 2 treatment.

These surprising effects are not evident since anxiolytic compounds either have no (i.e. buspirone) or deleterious (i.e. diazepam) effect on memory.

From therapeutic point of view the advantageous effect of compound 5-[2-[4-(2,3-dihydro-benzo[1,4]dioxine-5-yl)-piperazine-1-yl]-ethylamine]-2H-pyridazine-3-one falling under the general Formula (I) on learning and memory signifies that the compounds could be appropriate for treating and/or preventing diseases or conditions accompanying diseases wherein learning or memory functions are suffering a loss or there is a possibility to suffering a loss. Such diseases are, but not limited to—as mentioned earlier—Alzheimer's disease, Korsakoff syndrome, Huntington's disease, Parkinson disease and mental decline due to ageing processes or impairment of the cognitive functions due to exposure to toxic substances as well.

The daily dose of the compound of the general Formula I depends on the mode of administration, the body weight, age and condition of the patient to be treated, the severeness of the disease to be treated etc. The daily dose of the compounds of the general Formula I in indications defined is generally between 0.5 mg/kg and 150 mg/kg, preferably about 1-150 mg/kg, particularly preferably between about 10 mg/kg and 150 mg/kg.

Further details of the present invention are to be found in the following Examples without limiting the scope of protection to said Examples.

### EXAMPLE 1

### Preparation of 4-(3-((2-(2,3-dihydro-benzo[1,4]dioxine-5-yloxy)-ethyl)-methyl-amino)-propyl-amino)-5-chloro-2H-pyridazine-3-one oxalate

A mixture of 2.66 g (0.01 mole) of 4-(3-bromo-propylamino)-5-chloro-2H-pyridazine-3-one, 2.51 g (0.012 mole) of (2-(2,3-dihydro-benzo[1,4]dioxine-5-yloxy)-ethyl-methyl-amine, 2.8 ml (0.02 mole) of triethyl-amine and 40 ml of acetone is refluxed for 120 hours under stirring. The reaction mixture is cooled back, filtered and evaporated in vacuu. The residue is subjected to chromatography on a silica column and eluted with a 1:1:2 mixture of acetone/ethylacetate/chloroform. The fractions containing the desired compound are collected, evaporated and re-dissolved in a 15:1 mixture of diethylether and ethyl acetate.

To the solution a diethylether solution of oxalic acid is added drop-wise at room temperature under stirring. The precipitated crystals are filtered and washed with diethylether.

Thus 2.76 g of the desired compound are obtained. Yield: 57.0%. M.p.: 115-117° C.

Elementary analysis for the Formula C₂₀H₂₅ClN₄O₈ (484.90):

calc.: C, 49.54%; H, 5.20%; Cl, 7.31%; N, 11.55%.

found: C, 49.04%; H, 5.11%; Cl, 7.18%; N, 11.42%.

IR (KBr): 3300, 1720, 1640, 1610, 1114.

¹H-NMR (DMSO-d₆, i400): 12.8 (b, 1H), 7.60 (s, 1H), 6.77 (bt, J=6.7 Hz, 1H), 6.74 (t, J=8.2 Hz, 1H), 6.60 (dd, J1=1.5 Hz, J2=8.3 Hz, 1H), 6.53 (dd, J1=1.4 Hz, J2=8.2 Hz, 1H), 4.27 (t, J=5.1 Hz, 2H), 4.22 (s, 4H), 3.69 (˜q, J=6.7 Hz, 2H), 3.38 (t, J=5.0 Hz, 2H), 3.10 (t, J=7.7 Hz, 2H), 2.78 (s, 3H), 1.95 (m, 2H).

### EXAMPLE 2

### Preparation of 4-(4-(4-(2,3-dihydro-benzo[1,4]dioxine-5-yl)-piperazin-1-yl)-buthylamino)-5-chloro-2H-pyridazine-3-one

A mixture of 1.65 g (0.01 mole) of 4,5-dichloro-2H-pyridazine-3-one, 7.28 g (0.025 mole) of 4-(4-(2,3-dihydro-benzo[1,4]dioxine-5-yl)-piperazine-1-yl)-buthylamine and 40 ml of dioxane is refluxed for 24 hours under stirring. The reaction mixture is evaporated in vacuu. The residue is dissolved in toluene and extracted with a 10% sodium carbonate solution and water several times. The organic phase is dried over magnesium sulfate, filtered and the mother-lye is evaporated in vacuu. The residue is subjected to chromatography on a silica column and eluted with a 3:2:0.5 mixture of hexane/acetone/methanol. The fractions containing the desired compound are collected and evaporated. The residue is treated with diethylether and the crystals are filtered.

Thus 1.91 g of the desired compound are obtained.

Yield: 45.6%. M.p.: 160-162° C.

Elementary analysis for the Formula C₂₀H₂₅ClN₅O₃ (419.92):

calc.: C, 57.21%; H, 6.24%; Cl, 8.44%; N, 16.68%.

found: C, 57.26%; H, 6.32%; Cl, 8.33%; N, 16.49%.

IR (KBr): 3345, 1648, 1613.

¹H-NMR (CDCl₃, i400): 11.02 (bs, 1H), 7.52 (s, 1H), 6.77 (t, 1H, J=8.1 Hz), 6.59 (dd, 1H, J1=1.4 Hz, J2=8.2 Hz), 6.54 (dd, 1H, J1=1.5 Hz, J2=8.0 Hz), 5.89 (m, 1H), 4.28 (m, 4H), 3.77 (˜q, 2H, J=6.7 Hz), 3.11 (m, 4H), 2.67 (m, 4H), 2.46 (t, 2H, J=7.0 Hz), 1.68 (m, 4H).

### EXAMPLE 3

### Preparation of 5-{2-[4-(2,3-dihydro-1,4-benzodioxine-5-yl)-piperazine-1-yl]-ethylamino}-2H-pyridazine-3-one

Into a pressure-proof hydrogenating apparatus 3.9 g (0.01 mole) of 5-{2-[4-(2,3-dihydro-1,4-benzodioxine-5-yl)-piperazine-1-yl]-ethylamino}-4-chloro-2H-piridazine-3-one, 400 ml of a 9:1 mixture of methanol and distilled water, 0.45 g (0.0112 mole) of sodium hydroxide and 4 g of a palladium-charcoal catalyst (palladium content 8%) are weighed in. The reaction mixture is stirred at room temperature under a hydrogen pressure of 10 atm for 3 hours. The hydrogen is removed and the reaction mixture is refluxed for 5 minutes. The mixture is filtered until hot and the palladium-charcoal catalyst is washed three times with 33 ml of a 1:1 methanol/dichloromethane mixture each. The united mother-lye is evaporated to 30 ml. The residue is stirred under cooling with ice-cold water for half an hour. The precipitated crystals are filtered and washed with 10 ml of cooled methanol. The product is dried over phosphorous pentoxide at 140° C. for 3 hours.

Thus 2.92 g of the desired compound are obtained.

Yield: 81.7%. M.p.: 244-246° C.

Elementary analysis for the Formula C₁₈H₂₃N₅O₃ (357.42):

calc.: C, 60.49%; H, 6.49%; N, 19.59%.

found: C, 60.33%; H, 6.44%; N, 19.46%.

IR (KBr): 3325, 3277, 1612.

¹H-NMR (CDCl₃, i400): 11.85 (bs, 1H), 7.44 (d, J=2.1 Hz, 1H), 6.80 (bt, 1H), 6.66 (at, J=8.1 Hz, 1H), 6.44 (d, J=8.2 Hz, 1H), 6.41 (d, J=8.1 Hz, 1H), 5.35 (˜s, 1H), 4.16 (m, 2H), 3.08 (˜q, J=5.4 Hz, 2H), 2.92 (m, 4H), 2.51 (m, 6H).

¹³C-NMR (CDCl₃, i400): 162.31, 149.38, 143.99, 141.75, 136.34, 131.65, 120.48, 111.19, 110.33, 94.32, 63.98, 63.88, 55.91, 53.13, 50.16, 39.15.

Hydrochloride Salt:

IR (I (KBr): 32505, 2591, 1085.

¹H-NMR (DMSO-d₆, i400): 12.04 (bs, 1H), 11.33 (bs, 1H), 7.49 (m, 1H), 6.76 (t, J=8.1 Hz, 1H), 6.58 (dd, J1=1.2 Hz, J2=8.2 Hz, 1H), 6.52 (dd, J1=1.1 Hz, J2=7.9 Hz, 1H), 5.62 (d, J=2.3 Hz, 1H), 4.25 (m, 2H), 4.23 (m, 2H), 3.7-3.0 (m, 12H)

¹³C-NMR (DMSO-d₆, i400): 162.31, 148.86, 144.15, 140.02, 136.30, 131.55, 120.65, 112.14, 110.59, 95.44, 64.12, 63.92, 53.29, 51.42, 47.06, 36.19.

### EXAMPLE 4

### Preparation of 5-{2-[4-(methoxy-trifluoromethyl-phenyl)-piperazine-1-yl]-ethylamino}-2H-pyridazine-3-one trihydrochloride

Into a pressure-proof hydrogenating apparatus 3.7 g (0.0086 mole) of 5-{2-[4-(methoxy-trifluoromethyl-phenyl)-piperazine-1-yl]-ethylamino}-4-chloro-2H-pyridazine-3-one, 370 ml of methanol, 3.2 ml (0.018 mole) of diisopropyl-ethyl-amine and 3.7 g of a 8% palladium-charcoal catalyst are weighed in. The reaction mixture is stirred at room temperature under a hydrogen pressure of 10 atm for 4 hours. The hydrogen is removed. The reaction mixture is refluxed for 5 minutes, filtered until hot and the catalyst is washed three times with 30 ml of a 1:1 methanol/dichloromethane mixture each. The united mother-lies are evaporated. The residue is subjected to chromatography on a silica column and eluted with a 19:1 mixture of chloroform and methanol. The fractions which contain the product are evaporated. The residue is dissolved in a mixture of ethylacetate and diethylether and to the solution ether containing hydrogen chloride is added drop-wide. The precipitated crystals are stirred under cooling with ice-cold water for half an hour, filtered and washed in diethylether. The product is dried over phosphorous pentoxide at 80° C. for 3 hours.

Thus 1.84 g of the desired compound are obtained.

Yield: 54%. M.p.: 238-240° C.

Elementary analysis for the Formula C₁₈H₂₅Cl₃F₃N₅O₂ (506.79):

calc.: C, 42.66%; H, 4.97%; N, 13.82%; Cl, 20.99%.

found: C, 42.53%; H, 5.01%; N, 13.63%; Cl, 20.69%.

IR (KBr): 3294, 2340, 1630, 1330, 1115.

¹H-NMR (DMSO-d₆, i400): 13.23 (b, 1H), 11.49 (b, 1H), 8.43 (b, 1H), 7.90 (bs, 1H), 7.40 (d, J=8.5 Hz, 1H), 7.18 (d, J=8.7 Hz, 1H), 7.15 (s, 1H), 6.05 (bs, 1H), 3.89 (s, 3H), 3.13-3.75 (m, 12H).

¹³C-NMR (DMSO-d₆, i400): 162.14, 154.81, 150.30, 139.98, 134.04, 124.68 (q, J=271.6 Hz), 121.51 (q, J=31.7 Hz), 120.92 (q), 114.81 (q), 112.22, 93.60, 56.13, 53.09, 51.30, 46.69, 36.49.

What we claim is:

1. Use of the compounds of the general Formula 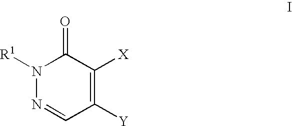
(wherein
R¹ stands for hydrogen or lower alkyl;
one of symbols X and Y stands for hydrogen or halogen and the other represents a group of the general Formula 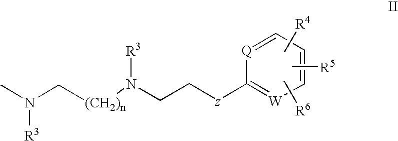
R² is hydrogen or lower alkyl;
n is 1, 2 or 3;
R³ is hydrogen, lower alkyl or aryl-lower alkyl;
Z is —O—; or
R³ and Z together with the intermediate atoms form a piperazino ring;
Q and W independently from each other stands for —CH═ or —N═; and
R⁴, R⁵ and R⁶ can be the same or different and stand for hydrogen, halogen, trifluoromethyl or lower alkoxy;
or R⁴ and R⁵ together form an ethylenedioxy group)
and pharmaceutically acceptable salts thereof for the preparation of pharmaceutical compositions suitable for the treatment and/or prophylaxis of malfunctions of memory and/or cognitive decline or prevention of decline of learning abilities.

2. Use according to claim 1 for the preparation of pharmaceutical compositions suitable for the treatment or prophylaxis of Korsakoff syndrome, Alzheimer disease, Huntington syndrome or Parkinson disease and/or mental decline due to ageing processes or impairment of the cognitive functions due to exposure to toxical substances.

3. Use according to claim 1 or 2 which comprises using as active ingredient a compound of the general Formula I or a pharmaceutically acceptable acid addition salt thereof in which
R¹ is hydrogen, methyl, ethyl or tertiary butyl;
one of symbols X and Y is hydrogen or chlorine and the other represents a group of the general Formula II;
R² is hydrogen or methyl;
n is 1 or 2;
R³ is hydrogen, methyl or benzyl;
Z is —O—; or
R³ and Z together with the intermediate atoms form a piperazino ring;
R⁴, R⁵ and R⁶ can be the same or different and stand for hydrogen or halogen; or R⁴ and R⁵ together form an ethylenedioxy group); and
Q and W stand for —CH═.

4. Use according to claim 1 which comprises using as active ingredient 4-(3-((2-(2,3-dihydro-benzo[1,4]dioxine-5-yloxy)-ethyl)-methyl-amino)-propyl-amino)-5-chloro-2H-pyridazine-3-one or a pharmaceutically acceptable salt thereof.

5. Use according to claim 1 which comprises using as active ingredient 4-(3-{[2-(2,3-dihydro-benzo[1,4]dioxine-5-yloxy)-ethyl]-propyl-amino}-propylamino)-5-chloro-2H-pyridazine-3-one or a pharmaceutically acceptable salt thereof.

6. Use according to claim 1 which comprises using as active ingredient 4-(3-(benzil-(2-(2,3-dihydro-benzo[1,4]dioxine-5-yloxy)-ethyl)-amino)-propyl-amino)-5-chloro-2H-pyridazine-3-one or a pharmaceutically acceptable salt thereof.

7. Use according to claim 1 which comprises using as active ingredient 4-(4-(4-(2,3-dihydro-benzo[1,4]dioxine-5-yl)-piperazine-1-yl)-butilamino)-5-chloro-2H-pyridazine-3-one or a pharmaceutically acceptable salt thereof.

8. Use according to claim 1 which comprises using as active ingredient 5-(2-(4-(2,3-dihydro-benzo[1,4]dioxine-5-yl)-piperazine-1-yl)-ethyl-amino)-4-chloro-2H-pyridazine-3-one or a pharmaceutically acceptable salt thereof.

9. Use according to claim 1 which comprises using as active ingredient 4-chloro-5-(2-(4-(2,3-dihydro-1,4-benzodioxine-5-yl)-piperazine-1-yl)-ethylamino)-2-methyl-2H-pyridazine-3-one or a pharmaceutically acceptable salt thereof.

10. Use according to claim 1 which comprises using as active ingredient 4-chloro-5-((2-(4-(2,3-dihydro-benzo[1,4]dioxine-5-yl)-piperazine-1-yl)-ethyl)-methyl-amino-2H-pyridazine-3-one or a pharmaceutically acceptable salt thereof.

11. Use according to claim 1 which comprises using as active ingredient 2-tert.-butil-5-chloro-4-(2-(4-(2,3-dihydro-benzo[1,4]dioxine-5-yl)-piperazine-1-yl)-ethylamino)-2H-pyridazine-3-one or a pharmaceutically acceptable salt thereof.

12. Use according to claim 1 which comprises using as active ingredient 4-(3-(2-(2,3-dihydro-benzo[1,4]dioxine-5-yloxy)-ethylamino)-propylamino)-2H-pyridazine-3-one or a pharmaceutically acceptable salt thereof.

13. Use according to claim 1 which comprises using as active ingredient -{2-[4-(2,3-dihydro-1,4-benzodioxine-5-yl)-piperazine-1-yl]-ethyl-amino}-2H-pyridazine-3-one or a pharmaceutically acceptable salt thereof.

14. Use according to claim 1 which comprises using as active ingredient 5-{2-[4-(7-chloro-2,3-dihydro-benzo[1,4]dioxine-5-yl)-piperazine-1-yl]-ethylamino}-2H-pyridazine-3-one or a pharmaceutically acceptable salt thereof.

15. Use according to claim 1 which comprises using as active ingredient 5-{3-[4-(2,3-dihydro-1,4-benzodioxine-5-yl)-piperazine-1-yl]-propylamino}-2H-pyridazine-3-one or a pharmaceutically acceptable salt thereof.

16. Use according to claim 1 which comprises using as active ingredient -(2-(2-(2,3-dihydro-benzo[1,4]dioxine-5-yloxy)-ethylamino)-ethyl-amino)-2H-pyridazine-3-one or a pharmaceutically acceptable salt thereof.

17. Use according to claim 1 which comprises using as active ingredient 5-{2-[4-(2,3-dihydro-1,4-benzodioxine-5-yl)-piperazine-1-yl]-ethyl-amino}-2-methyl-2H-pyridazine-3-one or a pharmaceutically acceptable salt thereof.

18. Use according to claim 1 which comprises using as active ingredient 5-({2-[4-(2,3-dihydro-benzo[1,4]dioxine-5-yl)-piperazine-1-yl]-ethyl}-methyl-amino)-2H-pyridazine-3-one hydrochloride or monohydrate thereof or a pharmaceutically acceptable salt thereof.

19. Use according to claim 1 which comprises using as active ingredient 5-(2-(4-(2,3-dihydro-benzo[1,4]dioxine-5-yl)piperazine-1-yl)-ethyl-methylamino)-2-methyl-2H-pyridazine-3-one or a pharmaceutically acceptable salt thereof.

20. Use according to claim 1 which comprises using as active ingredient 5-({2-[4-(2,3-dihydro-benzo[1,4]dioxine-5-yl)-piperazine-1-yl]-ethyl}-methyl-amino)-4-chloro-2-methyl-2H-pyridazine-3-one or a pharmaceutically acceptable salt thereof.

21. Use according to claim 1 which comprises using as active ingredient 5-(2-{benzyl-[2-(2,3-dihydro-benzo[1,4]dioxine-5-yloxy)-ethyl]-amino}-ethylamino)-4-chloro-2-methyl-2H-pyridazine-3-one or a pharmaceutically acceptable salt thereof.

22. Use according to claim 1 which comprises using as active ingredient 5-{2-[2-(2,3-dihydro-benzo[1,4]dioxine-5-yloxy)-ethylamino]-ethyl-amino}-2-methyl-2H-pyridazine-3-one or a pharmaceutically acceptable salt thereof.

23. Use according to claim 1 which comprises using as active ingredient 5-{2-[4-(metoxi-trifluoromethyl-phenyl)-piperazine-1-yl]-ethylamino}-2H-pyridazine-3-one or a pharmaceutically acceptable salt thereof.

24. Use according to claim 1 which comprises using as active ingredient 5-(2-[4-(2-fluoro-phenyl)-piperazine-1-yl]-ethylamino}-2H-pyridazine-3-one or a pharmaceutically acceptable salt thereof.

25. Use according to claim 1 which comprises using as active ingredient 5-(2-[4-phenyl-piperazine-1-yl]-ethylamino}-2H-pyridazine-3-one or a pharmaceutically acceptable salt thereof.

26. Use according to claim 1 which comprises using as active ingredient 5-[2-(4-piridin-2-yl-piperazine-1-yl)-ethylamino]-2H-pyridazine-3-one or a pharmaceutically acceptable salt thereof.

27. Use according to claim 1 which comprises using as active ingredient 5-[2-(4-pyrimidine-2-yl-piperazine-1-yl)-ethylamino]-2H-pyridazine-3-one or a pharmaceutically acceptable salt thereof.

28. Use according to claim 1 which comprises using as active ingredient 5-{2-[4-(3-chloro-phenyl)-piperazine-yl]-ethylamino}-2H-pyridazine-3-one or a pharmaceutically acceptable salt thereof.

29. Use according to claim 1 which comprises using as active ingredient 5-{2-[4-(4-fluoro-phenyl)-piperazine-1-yl]-ethylamino)}-2H-pyridazine-3-one or a pharmaceutically acceptable salt thereof.

30. Pharmaceutical composition suitable for the treatment and/or prophylaxis of malfunctions of memory and/or cognitive decline or prevention of decline of learning abilities comprising as active ingredient a compound of the general Formula I (wherein the substituents are as stated in claim 1) or a pharmaceutically acceptable acid addition salt thereof in admixture with suitable inert solid or liquid pharmaceutical carriers and/or auxiliary agents.

31. Process for the treatment or prophylaxis of malfunctions of memory and/or cognitive decline or prevention of decline of learning abilities which comprises administering to the patient in need of such treatment a pharmaceutically efficient amount of the compound of the general Formula I or an acid addition salt thereof.
